(19) United States
(12) Patent Application Publication
(10) Pub. No.: US 2006/0094679 A1
(43) Pub. Date: May 4, 2006

(54) Method of preventing or treating liver disease

(75) Inventors: Hirohito Tsubouchi, Kagoshima (JP); Satoru Hasuike, Miyazaki (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: MIYAZAKI PREFECTURE, Miyazaki-shi (JP)

(21) Appl. No.: 11/208,544

(22) Filed: Aug. 23, 2005

(30) Foreign Application Priority Data
Nov. 1, 2004 (JP) 318285/2004

(51) Int. Cl. A61K 48/00; A61K 38/17

(52) U.S. Cl. 514/44; 514/12

(57) ABSTRACT

An object of the present invention is to provide a method of preventing or treating liver disease, particularly non-alcoholic fatty liver disease, hepatic fibrosis, cirrhosis, or liver cancer. The present invention is directed to a method of preventing or treating liver disease comprising administering to a patient in need thereof an effective amount of an osteoactivin-like protein or a fragment thereof having liver disease-suppressing activity similar to that of osteoactivin or a gene comprising a DNA encoding such a protein or fragment.

### TECHNICAL FIELD

The present invention relates to a method of preventing or treating liver disease, particularly non-alcoholic fatty liver disease, hepatic fibrosis, cirrhosis, or liver cancer, which comprises administering an osteoactivin-like protein or a gene encoding the same as an active ingredient.

### BACKGROUND ART

Amelioration of obesity by dietetic therapy and by ergotherapy are thought to be effective therapeutic methods against non-alcoholic fatty liver disease. However in most cases, it is difficult to actually produce effects by the use of such therapies. Although there are Chinese medicines and liver-function-improving medicines as internal medicines, the effects thereof are often insufficient.

Antiviral agents are effectively used in therapeutic methods for disease due to cirrhosis or hepatic fibrosis, when the disease is caused by hepatitis B virus or hepatitis C virus. However, depending on pathological conditions, antiviral agents sometimes cannot be used due to side effects. Furthermore, there have been no effective therapeutic methods for overcoming disease due to cirrhosis or hepatic fibrosis, which is chronic liver disease not caused by hepatitis B virus or hepatitis C virus.

An example of a therapeutic method for liver cancer is a method that involves suppressing the growth of liver cancer or suppressing carcinogenesis by the use of an anticancer agent. However, the effect of such a method is not high and the use of such an agent is significantly limited due to side effects. In the meantime, therapeutic methods for liver cancer have been advanced. For example, there are therapeutic methods for liver cancer that involve operation, radiofrequency cauterization, and the like. However, these therapeutic methods are unable to suppress carcinogenesis. Patients that can be treated by such therapies are limited. Antiviral agents are effective in suppressing carcinogenesis due to hepatitis B virus or hepatitis C virus. However, agents cannot be administered to many patients due to side effects. Furthermore, in the case of liver cancer not caused by hepatitis B virus or hepatitis C virus, no effective carcinogenesis-suppressing agents exist.

In the meantime, osteoactivin is a protein identified from the osteoblasts of rats with osteopetrosis. It has been reported by Safadi et al. that osteoactivin has an effect on the differentiation of osteoblasts and bone matrix formation (Specification of U.S. Patent Application No. 2002/0151486; and Safadi, Fayez et al “Cloning and characterization of osteoactivin, a novel cDNA expressed in osteoblasts.” J of Cellular Biochemistry, 84 [1] 12-26, (2001)). Furthermore, the present inventors have studied the relationship between liver functions and osteoactivin (Onaga, Masaaki et al “Osteoactivin expressed during cirrhosis development in rats fed a choline-deficient, L-amino acid-defined diet, accelerates motility of hepatoma cells.” J of Hepatology, 39 [5] 7779-785 (2003) (Onaga et al. 1); and Onaga, Masaaki et al “Osteoactivin, a gene isolated from the liver of rat fed with a choline-deficient, L-amino acid defined diet, accelerated invasion and metastasis of hepatoma cells.” Hepatology, 34 [4] Pt.2, 384A (2001) Print Meeting Info.: 52nd Annual Meeting and Postgraduate Courses of the American Association for the Study of Liver Diseases. Dallas, Tex., USA. Nov. 9-13, 2001 (Onaga et al. 2)). Onaga et al. 1 reports that osteoactivin is expressed in liver cells of rats fed on a choline-deficient and L-amino acid-defined (CDAA) diet.

It has been unknown that osteoactivin has action to treat liver disease. In addition, U.S. Patent Application No. 2002/0151486 discloses the nucleic acid sequence of osteoactivin and therapeutic compositions containing osteoactivin. However, the document does not mention the action of osteoactivin to treat liver disease.

### SUMMARY OF THE INVENTION

An object of the present invention is to provide a method of preventing or treating liver disease, particularly non-alcoholic fatty liver disease, hepatic fibrosis, cirrhosis, or liver cancer.

As a result of intensive studies to achieve the above object, the present inventors have discovered that, surprisingly, an osteoactivin-like protein or a gene thereof isolated from the livers of rats fed on a choline-deficient and L-amino acid-defined diet is effective for preventing or treating non-alcoholic fatty liver disease, hepatic fibrosis, cirrhosis, or liver cancer. Thus the present inventors have completed the present invention.

Based on such findings, the present invention provides, as a new application of an osteoactivin-like protein or the gene thereof, a method of preventing or treating liver disease, particularly non-alcoholic fatty liver disease, hepatic fibrosis, cirrhosis, or liver cancer.

The present invention encompasses the following invention.
(1) A method of preventing or treating liver disease comprising administering to a patient in need thereof an effective amount of a gene comprising a DNA encoding an osteoactivin-like protein or a fragment thereof having liver disease-suppressing activity similar to that of osteoactivin.
(2) The method of preventing or treating liver disease of (1), wherein the DNA is the following (a) or (b):
(a) a DNA consisting of the nucleotide sequence of SEQ ID NO: 1, 3, 5, 7, or 9, or a partial sequence thereof; or
(b) a DNA hybridizing under stringent conditions to a DNA consisting of a nucleotide sequence complementary to that of the DNA of (a), which encodes a protein or a fragment thereof having liver disease-suppressing activity similar to that of osteoactivin.

These DNAs are used independently or in combination of 2 or more types thereof.
(3) A method of preventing or treating liver disease comprising administering to a patient in need thereof an effective amount of an osteoactivin-like protein or a fragment thereof having liver disease-suppressing activity similar to that of osteoactivin.
(4) The method of preventing or treating liver disease of (3), wherein the osteoactivin-like protein is the following (a) or (b):
(a) a protein consisting of the amino acid sequence of SEQ ID NO: 2, 4, 6, 8, or 10; or
(b) a protein consisting of an amino acid sequence derived from the amino acid sequence of SEQ ID NO: 2, 4, 6, 8, or 10 by deleting one or several amino acids in the amino acid sequence of SEQ ID NO: 2, 4, 6, 8, or 10, substituting another one or several amino acids for one or several amino acids in the amino acid sequence of SEQ ID NO: 2, 4, 6, 8, or 10, or adding another one or several amino acids to the amino acid sequence of SEQ ID NO: 2, 4, 6, 8, or 10; said protein (b) having liver disease-suppressing activity similar to that of osteoactivin.

These proteins or fragments thereof are used independently or in combination of 2 or more types thereof.
(5) The method of preventing or treating liver disease of (1) or (3), wherein the liver disease is at least 1 type of liver disease selected from the group consisting of non-alcoholic fatty liver disease, hepatic fibrosis, cirrhosis, and liver cancer.

### EFFECTS OF THE INVENTION

According to the present invention, a method of preventing or treating liver disease, particularly non-alcoholic fatty liver disease, hepatic fibrosis, cirrhosis, or liver cancer, is provided.

This description includes part or all of the contents as disclosed in the description of Japanese Patent Application No. 2004-318285, which is a priority document of the present application.

### PREFERRED EMBODIMENTS OF THE INVENTION

In the present invention, “osteoactivin-like protein” indicates osteoactivin or a protein having activity similar to, or preferably the same in quality as, that of osteoactivin. Specific examples of such osteoactivin-like protein include, in addition to osteoactivin, proteins such as GPNMB and DC-HIL. Furthermore, polypeptide fragments consisting of partial sequences of the amino acid sequences of these proteins and having activity similar to, or preferably the same in quality as, that of osteoactivin can also be used in the present invention. Hereinafter in this specification, the osteoactivin-like protein or a fragment thereof may be simply referred to as “osteoactivin-like protein.” Here, “activity that is the same in quality as that of osteoactivin” means liver disease-suppressing activity. Examples of “liver disease-suppressing activity” include activity of suppressing fatty liver, activity of suppressing hepatic fibrosis, and activity of suppressing a precancerous change in the liver. The activity of suppressing fatty liver can be evaluated based on activity of reducing lipid droplets in liver tissue. The activity of suppressing hepatic fibrosis can be evaluated based on activity of reducing a fibrosis area in a sliced sample of liver tissue. The activity of suppressing a precancerous change in the liver can be evaluated based on activity of reducing a GST-P-positive nodule area in a slice of liver tissue. In addition, “(activity that is) the same in quality” means that such activity is characteristically the same as liver disease-suppressing activity. Hence, liver disease-suppressing activity may vary in terms of strength of activity. Furthermore, quantitative aspects of the osteoactivin-like protein or a fragment thereof, such as its molecular weight, may be different from that of osteoactivin. In a preferable embodiment of the present invention, the osteoactivin-like protein or a fragment thereof has the same activity as that of osteoactivin in terms of strength as well as quality.

Source organisms from which such osteoactivin-like protein that can be used in the present invention is derived are not specifically limited. The osteoactivin-like protein is typically derived from mammals such as rats, mice, or humans.

A typical example of such osteoactivin-like protein is a protein consisting of the amino acid sequence of SEQ ID NO: 2 (rat osteoactivin), 4 (mouse DC-HIL), 6 (mouse GPNMB), 8 (human GPNMB), or 10 (rat GPNMB). Furthermore, examples of protein fragments that can be used in the present invention include a protein fragment consisting of the amino acid sequence ranging from the 23^{rd} to 572^{nd} amino acids of SEQ ID NO: 2 and a protein fragment consisting of the amino acid sequence ranging from the 23^{rd} to 560^{th} amino acids of SEQ ID NO: 8.

First, the present invention relates to a method of preventing or treating liver disease comprising administering to a patient in need thereof an effective amount of a DNA encoding the above osteoactivin-like protein.

A protein consisting of the amino acid sequence of SEQ ID NO: 2 (rat osteoactivin) is encoded by, for example, a DNA consisting of the nucleotide sequence ranging from the 115^{th} to 1833^{rd} nucleotides of SEQ ID NO: 1. A protein consisting of the amino acid sequence of SEQ ID NO: 4 (mouse DC-HIL) is encoded by, for example, a DNA consisting of the nucleotide sequence ranging from the 44^{th} to 1768^{th} nucleotides of SEQ ID NO: 3. A protein consisting of the amino acid sequence of SEQ ID NO: 6 (mouse GPNMB) is encoded by, for example, a DNA consisting of the nucleotide sequence ranging from the 80^{th} to 1804^{th} nucleotides of SEQ ID NO: 5. A protein consisting of the amino acid sequence of SEQ ID NO: 8 (human GPNMB) is encoded by, for example, a DNA consisting of the nucleotide sequence ranging from the 92^{nd} to 1774^{th} nucleotides of SEQ ID NO: 7. A protein consisting of the amino acid sequence of SEQ ID NO: 10 (rat GPNMB) is encoded by, for example, a DNA consisting of the nucleotide sequence ranging from the 58^{th} to 1776^{th} nucleotides of SEQ ID NO: 9. It is known by persons skilled in the art that these proteins can be encoded by sequences other than the nucleotide sequences specifically shown in the sequence listing because of degeneration of genetic codes.

A DNA, which hybridizes under stringent conditions to a DNA consisting of a nucleotide sequence complementary to that of the above DNA and encodes a protein having liver disease-suppressing activity similar to, or preferably the same in quality as, that of osteoactivin, can also be appropriately used in the present invention. Here, “stringent conditions” are, for example, conditions of 42° C., 50% formamide, 4×SSPE (1×SSPE=150 mM NaCl, 10 mM NaH₂PO₄.H₂O, 1 mM EDTA, and pH7.4), 5× Denhardt's solution, and 0.1% SDS; or conditions of a temperature between 60° C. and 68° C. and preferably between 55° C. and 68° C., and a sodium concentration between 250 mM and 350 mM and preferably between 300 mM and 400 mM.

The above DNA may be any of genomic DNA, cDNA, or synthetic DNA. As a means for cloning a DNA that completely encodes the osteoactivin-like protein, for example, a desired DNA is amplified from a DNA library or the like derived from the above source organism by a known PCR method using synthetic DNA primers having partial nucleotide sequences of the osteoactivin-like protein. DNA can also be amplified by an RT-PCR method using RNA fractions prepared from tissues and/or cells. The thus amplified DNA fragment can be cloned into an appropriate vector that can be amplified in a host such as Escherichia coli.

Second, the present invention relates to a method of preventing or treating liver disease comprising administering to a patient in need thereof an effective amount of the above osteoactivin-like protein. In the present invention, a protein consisting of an amino acid sequence derived from the amino acid sequence of the osteoactivin-like protein by deletion, substitution, or addition of 1 or several (for example, 1 to 10, preferably 1 to 7, more preferably 1 to 5, and most preferably 1 to 3) amino acids and has liver disease-suppressing activity can also be appropriately used.

The above protein may be any of a naturally derived protein, a chemically synthesized protein, or a recombinant protein prepared by a gene recombination technique.

A naturally derived protein can be isolated from cells or tissues expressing the protein by an appropriate combination of protein isolation and purification techniques. A chemically synthesized protein can be synthesized according to, for example, a chemical synthesis method such as a Fmoc method (fluorenylmethyloxycarbonyl method) or a tBoc method (t-butyloxycarbonyl method). Furthermore, the protein of the present invention can also be synthesized using various commercial peptide synthesizers. A recombinant protein can be produced by introducing a DNA having a nucleotide sequence encoding the protein into an appropriate expression system.

Examples of liver disease subjected to prevention or treatment by the present invention include non-alcoholic fatty liver disease, hepatic fibrosis, cirrhosis, and liver cancer. The preventive or therapeutic method of the present invention is useful for preventing or treating liver disease in mammals (e.g., human, rat, and mouse), and in particular, a human. The meanings of the term “treatment” or “therapy” in the present invention include not only curing such disease to recover normal conditions but also suppressing the progress of such disease (also referred to as “suppression”).

For use in the method of the present invention, an agent (hereinafter referred to as “gene therapeutic agent”) containing as an active ingredient a gene comprising a DNA encoding an osteoactivin-like protein can be prepared. The gene therapeutic agent can be caused to sufficiently exert its action by (a) administering the gene to a subject to express the gene or by (b) inserting the gene into a cell to express the gene and then transplanting the cell into a subject, so as to increase the level of the osteoactivin-like protein in the subject's tissues.

The gene therapeutic agent can be produced by a conventional method. Specifically, the agent can be produced by compounding an osteoactivin-like protein gene alone with or by inserting the gene into an appropriate vector such as a plasmid vector, a retrovirus vector, an adenovirus vector, or an adenovirus-associated virus vector and then compounding the vector with a base that is generally used for a gene therapeutic agent.

As the above base, a base that is generally used for an injection can be used. Examples of such base include distilled water, salt solutions of such as sodium chloride or a mixture of sodium chloride and inorganic salt, solutions of such as mannitol, lactose, dextran, and glucose, amino acid solutions of such as glycine and arginine, organic acid solutions, and mixed solutions of salt solutions and glucose solutions. Alternatively, according to a conventional method known by persons skilled in the art, an injection can be prepared as a solution, suspension, or a disperse solution using an aid such as an osmotic regulator, a pH adjustor, a vegetable oil, or a surfactant in these bases. These injections can also be prepared as pharmaceutical preparations that are dissolved before use by manipulation such as pulverization or freeze-drying.

Furthermore, the gene therapeutic agent can also be produced by adding a gene of interest into a liposome suspension, freezing the suspension, and then thawing the frozen product. Liposomes can be prepared by a conventional method. Liposomes having genes encapsulated therein can be directly suspended in water, physiological saline, or the like, and then intravenously administered.

The administration form for the gene therapeutic agent may be general whole-body administration such as intravenous administration or arterial administration, or may be local administration to the liver. For example, an administration form that is a combination of a catheter technique, a gene-transfer technique, surgical operation, and the like can be employed. In addition among the above administration forms, intravenous administration may be particularly preferable.

The dose of the above gene therapeutic agent for a patient who needs prevention or treatment for liver disease differs depending on a patient's age, sex, symptoms, administration route, number of administrations, and dosage form. Generally, the dose for an adult ranges from approximately 1 μg/kg body weight to 1000 mg/kg body weight in terms of the weight of the gene per day. The number of administrations is not particularly limited.

For use in the method of the present invention, an agent containing as an active ingredient the osteoactivin-like protein can be formulated according to a conventional method. For example, according to need, such agents can be administered orally in the form of sugar-coated or enteric coated tablets, capsules, elixirs, microcapsules, and the like. They can also be administered parenterally in the form of injections such as aseptic solutions or suspension agents prepared by mixing the agent with water or pharmaceutically acceptable solutions other than water. Moreover, various administration methods including direct administration to local regions can be used. A preferable form of pharmaceutical preparation is a tablet or an aqueous solution containing the osteoactivin-like protein. A preferable administration method is oral administration or administration by intravenous injection. In addition, among the above administration forms, administration by intravenous injection is particularly preferable. Furthermore, the preventive or therapeutic agent can be produced by admixing the osteoactivin-like protein with a physiologically acceptable carrier, flavoring agent, excipient, vehicle, antiseptic agent, stabilizer, binder, and the like, in a generally accepted unit dose form. Furthermore, the agent can also be used together with other ingredients that are useful as remedies. Examples of such other ingredients that can be used together with the agent include therapeutic agents for liver disease, such as antiviral agents (e.g., interferon), Strong Neo-Minophagen C and products analogous thereto, Chinese medicines, and vitamin preparations.

As additives that can be admixed into tablets, capsules, or the like, for example, binders such as gelatine, corn starch, gum tragacanth, or gum Arabic, excipients such as crystalline cellulose, swelling agents such as corn starch, gelatine, or alginic acid, lubricants such as magnesium stearate, sweetening agents such as sucrose, lactose, or saccharin, or flavoring agents such as peppermint, oil of Gaultheria adenothrix, or cherry may be used. When a unit form is a capsule, in addition to materials of the above types, liquid carriers such as fats and fatty oils may also be employed. Aseptic compositions for injection can be prescribed according to general implemented formulation, for example, such that active substances in vehicles such as water for injection and naturally produced plant oil such as sesame oil or coconut oil are dissolved or suspended. Examples of aqueous solutions for injection include physiological saline and isotonizing solutions (e.g., D-sorbitol, D-mannitol, and sodium chloride) containing glucose or other adjuvants. Such an aqueous solution may be used together with an appropriate solubilizing agent, such as alcohol (e.g., ethanol), polyalcohol (e.g., propylene glycol or polyethylene glycol), or a nonionic surfactant (e.g., polysorbate 80 (trade mark) or HCO-50). Examples of oil solutions include sesame oil and soybean oil. Oil solutions may be used together with benzyl benzoate, benzyl alcohol, or the like as solubilizing agents. Furthermore, such solutions may be compounded with buffer agents (e.g., phosphate buffer and sodium acetate buffer), soothing agents (e.g., benzalkonium chloride and procaine hydrochloride), stabilizers (e.g., human serum albumin and polyethylene glycol), preservatives (e.g., benzyl alcohol and phenol), antioxidants, and the like. Appropriate ampules are generally filled with the thus prepared injections.

The dose of the osteoactivin-like protein for a patient who needs prevention or treatment for liver disease differs depending on a patient's age, sex, and symptoms, the administration route, the number of administrations, and the dosage form. Generally, the dose for an adult ranges from approximately 0.001 μg/kg body weight to 1000 mg/kg body weight in terms of protein quantity per day. The number of administrations is not particularly limited.

The present invention will be further described specifically by referring to an example, but it is not limited by such example.

### EXAMPLE

Normal rats (control group) and transgenic rats (test group) that had been genetically altered to strongly express a rat osteoactivin gene only in the liver were fed on a choline-deficient and L-amino acid-defined (CDAA) diet for 12 weeks. The rats were evaluated for hepatic fibrosis, precancerous changes, and fatty liver.

SD rats (Japan SLC, Inc.) were used as normal rats (control group).

Transgenic rats were produced by micro-injecting the rat osteoactivin gene (SEQ ID NO: 1) under control of a human SAP gene promoter into fertilized eggs of SD rats. The expression levels of osteoactivin in the liver tissues of the thus obtained transgenic rats were confirmed using the RT-PCR method. It was confirmed that the expression levels of osteoactivin in the transgenic rats were at least 2 times greater than the levels in normal rats. Incidentally, for example, the following document discloses that in transgenic rats obtained by microinjection of a gene under control of an SAP gene promoter into rat fertilized eggs, a gene of interest is strongly expressed in the liver tissues: Miyazaki T, Ohura T, Kobayashi M, Shigematsu Y, Yamaguchi S, Suzuki Y, Hata I, Aoki Y, Yang X, Minjares C, Haruta I, Uto H, Ito Y, and Muller U. Fatal propionic acidemia in mice lacking propionyl-CoA carboxylase and its rescue by postnatal, liver-specific supplementation via a transgene. J Biol Chem. 2001. 21; 276 (38): 35995-9.

The CDAA diet used in this experiment had been purchased from Dyets Inc. (518752 Choline Deficient Diet; see the home page of this company regarding specific compositions: http://www.dyets.com/518753.htm). 6 rats of the control group and 6 rats of the test group were fed ad libitum on this diet for 12 weeks.

3 slices of sirius-red-stained samples and 3 slices of GST-P-stained samples were prepared from the right lobe of the liver of each rat by a conventional method.

Hepatic fibrosis was evaluated based on fibrosis areas. Images of 10 fields were randomly incorporated at ×100 magnification from each slice of the above sirius-red-stained rat liver samples. Fibrosis areas were measured using pixs 2000 Pro (INNOTECH CORPORATION), the ratio of each fibrosis area to background tissue area was calculated, and then the average of the results of 10 fields was calculated. As a result, the average in the control group was found to be 4.99±1.99, the average in the test group was found to be 1.29±0.09, and p value was found to be 0.009 (Mann-Whitney U-test). Hence, osteoactivin suppressed hepatic fibrosis.

Precancerous changes were evaluated based on GST-P-positive nodule areas (Area Occupied by Foci). For each slice of the above GST-P-stained rat liver samples, GST-P-positive nodule areas were measured using pixs 2000 Pro (INNOTECH CORPORATION). The ratio of each total GST-P-positive nodule area to liver tissue area was calculated and then the average of the results for 3 slices was calculated. As a result, the average in the control group was found to be 0.26±0.35, the average in the test group was found to be 0.02±0.02, and p value was found to be 0.016 (Mann-Whitney U-test). Hence, osteoactivin suppressed precancerous changes.

Fatty liver was evaluated by HE staining and microscopic examination of areas (proportions) occupied by lipid droplets. Whereas lipid droplets occupied approximately 80% of the liver tissue in the control group, lipid droplets occupied 40% or less of the liver tissue in the test group. Specifically, the results indicate that lipid droplets in the liver tissue were reduced by 50% or more through gene alteration and that osteoactivin can suppress non-alcoholic fatty liver disease.

All publications, patents, and patent applications cited herein are incorporated herein by reference in their entirety.

1. A method of preventing or treating liver disease comprising administering to a patient in need thereof an effective amount of a gene comprising a DNA encoding an osteoactivin-like protein or a fragment thereof having liver disease-suppressing activity similar to that of osteoactivin.

2. The method of preventing or treating liver disease according to claim 1, wherein the DNA is the following (a) or (b):
(a) a DNA consisting of the nucleotide sequence of SEQ ID NO: 1, 3, 5, 7, or 9, or a partial sequence thereof; or
(b) a DNA hybridizing under stringent conditions to a DNA consisting of a nucleotide sequence complementary to that of the DNA of (a), which encodes a protein or a fragment thereof having liver disease-suppressing activity similar to that of osteoactivin.

3. A method of preventing or treating liver disease comprising administering to a patient in need thereof an effective amount of an osteoactivin-like protein or a fragment thereof having liver disease-suppressing activity similar to that of osteoactivin.

4. The method of preventing or treating liver disease according to claim 3, wherein the osteoactivin-like protein is the following (a) or (b):
(a) a protein consisting of the amino acid sequence of SEQ ID NO: 2, 4, 6, 8, or 10; or
(b) a protein consisting of an amino acid sequence derived from the amino acid sequence of SEQ ID NO: 2, 4, 6, 8, or 10 by deleting one or several amino acids in the amino acid sequence of SEQ ID NO: 2, 4, 6, 8, or 10, substituting another one or several amino acids for one or several amino acids in the amino acid sequence of SEQ ID NO: 2, 4, 6, 8, or 10, or adding another one or several amino acids to the amino acid sequence of SEQ ID NO: 2, 4, 6, 8, or 10; said protein (b) having liver disease-suppressing activity similar to that of osteoactivin.

5. The method of preventing or treating liver disease according to claim 1, wherein the liver disease is at least 1 type of liver disease selected from the group consisting of non-alcoholic fatty liver disease, hepatic fibrosis, cirrhosis, and liver cancer.

6. The method of preventing or treating liver disease according to claim 3, wherein the liver disease is at least 1 type of liver disease selected from the group consisting of non-alcoholic fatty liver disease, hepatic fibrosis, cirrhosis, and liver cancer.


SEQUENCE LISTING (10 sequences)

SEQ ID NO: 1 - DNA, 2320 bp, Rattus norvegicus
  CDS (115)..(1833)
gtatttcataaaacagagaggatcgcaggaggccggcactctgactcctggtggatgggactagggagtcagagtcaagccctgactggctgagggcgggcgctccgagtcagcatggaaagtctctgcggggtcctggtatttctgctgctggctgcaggactgccgctccaggcggccaagcggttccgtgatgtgctgggccatgagcagtatccggatcacatgagggagaacaaccaattacgtggctggtcttcagatgaaaatgaatgggatgaacagctgtatccagtgtggaggaggggagagggcagatggaaggactcctgggaaggaggccgtgtgcaggcagccctaaccagtgattcaccggccttggtgggttccaatatcaccttcgtagtgaacctggtgttccccagatgccagaaggaagatgccaacggcaatatcgtctatgagaggaactgcagaagtgatttggagctggcttctgacccgtatgtctacaactggaccacaggggcagacgatgaggactgggaagacaacaccagccaaggccagcacctcaggttccccgacgggaagcccttccctcgcccccacggacggaagaaatggaacttcgtctacgtcttccacacacttggtcagtattttcaaaagctgggtcagtgttcagcacgagtttctataaacacagtcaacttgacagttggccctcaggtcatggaagtgattgtctttcgaagacacggccgggcatacattcccatctccaaagtgaaagacgtgtatgtgataacagatcagatccctatattcgtgaccatgtaccagaagaatgaccggaactcgtctgatgaaaccttcctcagagacctccccattttcttcgatgtcctcattcacgatcccagtcatttcctcaactactctgccatttcctacaagtggaactttggggacaacactggcctgtttgtctccaacaatcacactttgaatcacacgtatgtgctcaatggaaccttcaactttaacctcaccgtgcaaactgcagtgccgggaccatgcccctcacccacaccttcgccttcttcttcgacttctccttcgcctgcatcttcgccttcacccacattatcaacacctagtccctctttaatgcctactggctacaaatccatggagctgagtgacatttccaatgaaaactgccgaataaacagatatggttacttcagagccaccatcacaattgtagatggaatcctagaagtcaacatcatccaggtagcagatgtcccaatccccacactgcagcctgacaactcactgatggacttcattgtgacctgcaaaggggccactcccacggaagcctgtacgatcatctctgaccccacctgccagatcgcccagaacagggtgtgcagcccggtggctgtggatgagctgtgcctcctgtccgtgaggagagccttcaatgggtccggcacgtactgtgtgaatttcactctgggagacgatgcaagcctggccctcaccagcgccctgatctctatccctggcaaagacctaggctcccctctgagaacagtgaatggtgtcctgatctccattggctgcctggccatgtttgtcaccatggttaccatcttgctgtacaaaaaacacaagacgtacaagccaataggaaactgcaccaggaacgtggtcaagggcaaaggcctgagtgtttttctcagccatgcaaaagccccgttctcccgaggagaccgggagaaggatccactgctccaggacaagccatggatgctctaagtcttcactctcacttctgactgggaacccactcttctgtgcatgtatgtgagctgtgcagaagtacatgactggtagctgttgttttctacggattattgtaaaatgtatatcatggtttagggagtgtagttaattggcattttagtgaagggatgggaagacagtatttcttcgcatctgtattgtggtttttatactgttaatagggtgggcacattgtgtctgaagggggagggggaggtcactgctacttaaggtcctaggttaactgggagaggatgccccaggctccttagatttctacacaagatgtgcctgaacccagctagtcctgacctaaaggccatgcttcatcaactctatctcagctcattgaacatacctgagcgcctgatggaattataatggaaccaagcttgttgtatggtgtgtgtgtgtacataagatactcattaaaaagacagtctattaaaaaaaaaaaaaa

SEQ ID NO: 2 - protein, 572 aa, Rattus norvegicus
MESLCGVLVFLLLAAGLPLQAAKRFRDVLGHEQYPDHMRENNQLRGWSSDENEWDEQLYPVWRRGEGRWKDSWEGGRVQAALTSDSPALVGSNITFVVNLVFPRCQKEDANGNIVYERNCRSDLELASDPYVYNWTTGADDEDWEDNTSQGQHLRFPDGKPFPRPHGRKKWNFVYVFHTLGQYFQKLGQCSARVSINTVNLTVGPQVMEVIVFRRHGRAYIPISKVKDVYVITDQIPIFVTMYQKNDRNSSDETFLRDLPIFFDVLIHDPSHFLNYSAISYKWNFGDNTGLFVSNNHTLNHTYVLNGTFNFNLTVQTAVPGPCPSPTPSPSSSTSPSPASSPSPTLSTPSPSLMPTGYKSMELSDISNENCRINRYGYFRATITIVDGILEVNIIQVADVPIPTLQPDNSLMDFIVTCKGATPTEACTIISDPTCQIAQNRVCSPVAVDELCLLSVRRAFNGSGTYCVNFTLGDDASLALTSALISIPGKDLGSPLRTVNGVLISIGCLAMFVTMVTILLYKKHKTYKPIGNCTRNVVKGKGLSVFLSHAKAPFSRGDREKDPLLQDKPWML

SEQ ID NO: 3 - DNA, 2279 bp, Mus musculus
  CDS (44)..(1768)
gtcagagtcaagccctgactggttgcaggcgctcggagtcagcatggaaagtctctgcggggtcctgggatttctgctgctggctgcaggactgcctctccaggctgccaagcgatttcgtgatgtgctgggccatgaacagtatcccaatcacatgagagagcacaaccaattacgtggctggtcttcggatgaaaatgaatgggatgaacacctgtatccagtgtggaggaggggagacggcaggtggaaggactcctgggaaggaggccgtgtgcaggcagtcctgaccagtgactcaccggctctggtgggttccaatatcacctttgtggtgaacctggtgttccccagatgccagaaggaagatgctaatggcaatatcgtctatgagaagaactgcaggaatgatttgggactgacctctgacctgcatgtctacaactggactgcaggggcagatgatggtgactgggaagatggcaccagccgaagccagcatctcaggttcccggacaggaggcccttccctcgcccccatggatggaagaaatggagctttgtctacgtctttcacacacttggccagtatttccaaaaactgggtcggtgttcagcacgggtttctataaacacagtcaacttgacagctggccctcaggtcatggaagtgactgtctttcgaagatacggccgggcatacattcccatctcgaaggtgaaagatgtgtatgtgataacagatcagatccctgtattcgtgaccatgtcccagaagaatgacaggaacttgtctgatgagatcttcctcagagacctccccatcgtcttcgatgtcctcattcatgatcccagccacttcctcaacgactctgccatttcctacaagtggaactttggggacaacactggcctgtttgtctccaacaatcacactttgaatcacacttatgtgctcaatggaaccttcaaccttaacctcaccgtgcaaactgcagtgcccgggccatgccctcccccttcgccttcgactccgcctccaccttcaactccgccctcacctccgccctcacctctgcccacattatcaacacctagcccctctttaatgcctactggttacaaatccatggagctgagtgacatttccaatgaaaactgccgaataaacagatatggctacttcagagccaccatcacaattgtagaggggatcctggaagtcagcatcatgcagatagcagatgtccccatgcccacaccgcagcctgccaactccctgatggacttcactgtgacctgcaaaggggccacccccatggaagcctgtacgatcatctccgaccccacctgccagatcgcccagaaccgggtctgcagccctgtggctgtggatgggctgtgcctgctgtctgtgagaagagccttcaatgggtctggcacctactgtgtgaatttcactctgggagatgatgcaagcctggccctcaccagcaccctgatctctatccctggcaaagacccagactcccctctgagagcagtgaatggtgtcctgatctccattggctgcctggctgtgcttgtcaccatggttaccatcttgctgtacaaaaaacacaaggcgtacaagccaataggaaactgccccaggaacacggtcaagggcaaaggcctgagtgttctcctcagccacgcgaaagccccgttcttccgaggagaccaggagaaggatccattgctccaggacaagccaaggacactctaagtctttggccttccctctgaccaggaacccactcttctgtgcatgtatgtgagctgtgcagaagtatgtggctgggaactgttgttctctaaggattattgtaaaatgtatatcgtggcttagggagtgtggttaaatagcattttagagaagacatgggaagacttagtgtttcttcccatctgtattgtggtttttacactgttcgtgaggtggacacgctgtgtctgaaggggaggtggggtcactgctacttaaggtcctaggttaactgggggagataccacagatgcctcagctttccacataacatgggcatgaacccagctaatcaccacctgaaggccatgcttcatctgccttccaactcactgagcatgcctgagctcctgacaaaattataatgggcccgggctttgtgtatggtgcgtgtgtgtacatattctactcattaaaaaggtagtctaaaaaaaaaaaaaaaaaaaaaaaaaaaaaaaaaaaaaaaaaaaaa

SEQ ID NO: 4 - protein, 574 aa, Mus musculus
MESLCGVLGFLLLAAGLPLQAAKRFRDVLGHEQYPNHMREHNQLRGWSSDENEWDEHLYPVWRRGDGRWKDSWEGGRVQAVLTSDSPALVGSNITFVVNLVFPRCQKEDANGNIVYEKNCRNDLGLTSDLHVYNWTAGADDGDWEDGTSRSQHLRFPDRRPFPRPHGWKKWSFVYVFHTLGQYFQKLGRCSARVSINTVNLTAGPQVMEVTVFRRYGRAYIPISKVKDVYVITDQIPVFVTMSQKNDRNLSDEIFLRDLPIVFDVLIHDPSHFLNDSAISYKWNFGDNTGLFVSNNHTLNHTYVLNGTFNLNLTVQTAVPGPCPPPSPSTPPPPSTPPSPPPSPLPTLSTPSPSLMPTGYKSMELSDISNENCRINRYGYFRATITIVEGILEVSIMQIADVPMPTPQPANSLMDFTVTCKGATPMEACTIISDPTCQIAQNRVCSPVAVDGLCLLSVRRAFNGSGTYCVNFTLGDDASLALTSTLISIPGKDPDSPLRAVNGVLISIGCLAVLVTMVTILLYKKHKAYKPIGNCPRNTVKGKGLSVLLSHAKAPFFRGDQEKDPLLQDKPRTL

SEQ ID NO: 5 - DNA, 2299 bp, Mus musculus
  CDS (80)..(1804)
gccgacactgtgactcctggtggatgggactggggagtcagagtcaagccctgactggttgcaggcgctcggagtcagcatggaaagtctctgcggggtcctgggatttctgctgctggctgcaggactgcctctccaggctgccaagcgatttcgtgatgtgctgggccatgaacagtatcccaatcacatgagagagcacaaccaattacgtggctggtcttcggatgaaaatgaatgggatgaacacctgtatccagtgtggaggaggggagacggcaggtggaaggactcctgggaaggaggccgtgtgcaggcagtcctgaccagtgactcaccggctctggtgggttccaatatcacctttgtggtgaacctggtgttccccagatgccagaaggaagatgctaatggcaatatcgtctatgagaagaactgcaggaatgatttgggactgacctctgacctgcatgtctacaactggactgcaggggcagatgatggtgactgggaagatggcaccagccgaagccagcatctcaggttcccggacaggaggcccttccctcgcccccatggatggaagaaatggagctttgtctacgtctttcacacacttggccagtatttccaaaaactgggtcggtgttcagcacgggtttctataaacacagtcaacttgacagctggccctcaggtcatggaagtgactgtctttcgaagatacggccgggcatacattcccatctcgaaggtgaaagatgtgtatgtgataacagatcagatccctgtattcgtgaccatgtcccagaagaatgacaggaacttgtctgatgagatcttcctcagagacctccccatcgtcttcgatgtcctcattcatgatcccagccacttcctcaacgactctgccatttcctacaagtggaactttggggacaacactggcctgtttgtctccaacaatcacactttgaatcacacttatgtgctcaatggaaccttcaaccttaacctcaccgtgcaaactgcagtgcccgggccatgccctcccccttcgccttcgactccgcctccaccttcaactccgccctcacctccgccctcacctctgcccacattatcaacacctagcccctctttaatgcctactggttacaaatccatggagctgagtgacatttccaatgaaaactgccgaataaacagatatggctacttcagagccaccatcacaattgtagaggggatcctggaagtcagcatcatgcagatagcagatgtccccatgcccacaccgcagcctgccaactccctgatggacttcactgtgacctgcaaaggggccacccccatggaagcctgtacgatcatctccgaccccacctgccagatcgcccagaaccgggtctgcagccctgtggctgtggatgggctgtgcctgctgtctgtgagaagagccttcaatgggtctggcacctactgtgtgaatttcactctgggagatgatgcaagcctggccctcaccagcaccctgatctctatccctggcaaagacccagactcccctctgagagcagtgaatggtgtcctgatctccattggctgcctggctgtgcttgtcaccatggttaccatcttgctgtacaaaaaacacaaggcgtacaagccaataggaaactgccccaggaacacggtcaagggcaaaggcctgagtgttctcctcagccacgcgaaagccccgttcttccgaggagaccaggagaaggatccattgctccaggacaagccaaggacactctaagtctttggccttccctctgaccaggaacccactcttctgtgcatgtatgtgagctgtgcagaagtatgtggctgggaactgttgttctctaaggattattgtaaaatgtatatcgtggcttagggagtgtggttaaatagcattttagagaagacatgggaagacttagtgtttcttcccatctgtattgtggtttttacactgttcgtggggtggacacgctgtgtctgaaggggaggtggggtcactgctacttaaggtcctaggttaactgggggagataccacagatgcctcagctttccacataacatgggcatgaacccagctaatcaccacctgaaggccatgcttcatctgccttccaactcactgagcatgcctgagctcctgacaaaattataatgggcccgggctttgtgtatggtgcgtgtgtgtacatattctactcattaaaaaggtagtctaaaaaaaaaaaaaaaaaaaaaaaaaaaaa

SEQ ID NO: 6 - protein, 574 aa, Mus musculus; identical to SEQ ID NO: 4

SEQ ID NO: 7 - DNA, 2669 bp, Homo sapiens
  CDS (92)..(1774)
cagatgccagaagaacactgttgctcttggtggacgggcccagaggaattcagagttaaaccttgagtgcctgcgtccgtgagaattcagcatggaatgtctctactatttcctgggatttctgctcctggctgcaagattgccacttgatgccgccaaacgatttcatgatgtgctgggcaatgaaagaccttctgcttacatgagggagcacaatcaattaaatggctggtcttctgatgaaaatgactggaatgaaaaactctacccagtgtggaagcggggagacatgaggtggaaaaactcctggaagggaggccgtgtgcaggcggtcctgaccagtgactcaccagccctcgtgggctcaaatataacatttgcggtgaacctgatattccctagatgccaaaaggaagatgccaatggcaacatagtctatgagaagaactgcagaaatgaggctggtttatctgctgatccatatgtttacaactggacagcatggtcagaggacagtgacggggaaaatggcaccggccaaagccatcataacgtcttccctgatgggaaaccttttcctcaccaccccggatggagaagatggaatttcatctacgtcttccacacacttggtcagtatttccagaaattgggacgatgttcagtgagagtttctgtgaacacagccaatgtgacacttgggcctcaactcatggaagtgactgtctacagaagacatggacgggcatatgttcccatcgcacaagtgaaagatgtgtacgtggtaacagatcagattcctgtgtttgtgactatgttccagaagaacgatcgaaattcatccgacgaaaccttcctcaaagatctccccattatgtttgatgtcctgattcatgatcctagccacttcctcaattattctaccattaactacaagtggagcttcggggataatactggcctgtttgtttccaccaatcatactgtgaatcacacgtatgtgctcaatggaaccttcagccttaacctcactgtgaaagctgcagcaccaggaccttgtccgccaccgccaccaccacccagaccttcaaaacccaccccttctttaggacctgctggtgacaaccccctggagctgagtaggattcctgatgaaaactgccagattaacagatatggccactttcaagccaccatcacaattgtagagggaatcttagaggttaacatcatccagatgacagacgtcctgatgccggtgccatggcctgaaagctccctaatagactttgtcgtgacctgccaagggagcattcccacggaggtctgtaccatcatttctgaccccacctgcgagatcacccagaacacagtctgcagccctgtggatgtggatgagatgtgtctgctgactgtgagacgaaccttcaatgggtctgggacgtactgtgtgaacctcaccctgggggatgacacaagcctggctctcacgagcaccctgatttctgttcctgacagagacccagcctcgcctttaaggatggcaaacagtgccctgatctccgttggctgcttggccatatttgtcactgtgatctccctcttggtgtacaaaaaacacaaggaatacaacccaatagaaaatagtcctgggaatgtggtcagaagcaaaggcctgagtgtctttctcaaccgtgcaaaagccgtgttcttcccgggaaaccaggaaaaggatccgctactcaaaaaccaagaatttaaaggagtttcttaaatttcgaccttgtttctgaagctcacttttcagtgccattgatgtgagatgtgctggagtggctattaacctttttttcctaaagattattgttaaatagatattgtggtttggggaagttgaattttttataggttaaatgtcattttagagatggggagagggattatactgcaggcagcttcagccatgttgtgaaactgataaaagcaacttagcaaggcttcttttcattattttttatgtttcacttataaagtcttaggtaactagtaggatagaaacactgtgtcccgagagtaaggagagaagctactattgattagagcctaacccaggttaactgcaagaagaggcgggatactttcagctttccatgtaactgtatgcataaagccaatgtagtccagtttctaagatcatgttccaagctaactgaatcccacttcaatacacactcatgaactcctgatggaacaataacaggcccaagcctgtggtatgatgtgcacacttgctagactcagaaaaaatactactctcataaatgggtgggagtattttggtgacaacctactttgcttggctgagtgaaggaatgatattcatatattcatttattccatggacatttagttagtgctttttatataccaggcatgatgctgagtgacactcttgtgtatatttccaaatttttgtatagtcgctgcacatatttgaaatcatatattaagactttccaaagatgaggtccctggtttttcatggcaacttgatcagtaaggatttcacctctgtttgtaactaaaaccatctactatatgttagacatgacattctttttctctccttcctgaaaaataaagtgtgggaagagacaaaaaaaaaaaaa

SEQ ID NO: 8 - protein, 560 aa, Homo sapiens
MECLYYFLGFLLLAARLPLDAAKRFHDVLGNERPSAYMREHNQLNGWSSDENDWNEKLYPVWKRGDMRWKNSWKGGRVQAVLTSDSPALVGSNITFAVNLIFPRCQKEDANGNIVYEKNCRNEAGLSADPYVYNWTAWSEDSDGENGTGQSHHNVFPDGKPFPHHPGWRRWNFIYVFHTLGQYFQKLGRCSVRVSVNTANVTLGPQLMEVTVYRRHGRAYVPIAQVKDVYVVTDQIPVFVTMFQKNDRNSSDETFLKDLPIMFDVLIHDPSHFLNYSTINYKWSFGDNTGLFVSTNHTVNHTYVLNGTFSLNLTVKAAAPGPCPPPPPPPRPSKPTPSLGPAGDNPLELSRIPDENCQINRYGHFQATITIVEGILEVNIIQMTDVLMPVPWPESSLIDFVVTCQGSIPTEVCTIISDPTCEITQNTVCSPVDVDEMCLLTVRRTFNGSGTYCVNLTLGDDTSLALTSTLISVPDRDPASPLRMANSALISVGCLAIFVTVISLLVYKKHKEYNPIENSPGNVVRSKGLSVFLNRAKAVFFPGNQEKDPLLKNQEFKGVS

SEQ ID NO: 9 - DNA, 2282 bp, Rattus norvegicus
  CDS (58)..(1776)
ggactagggagtcagagtcaagccctgactggctgagggcgggcgctccgagtcagcatggaaagtctctgcggggtcctggtatttctgctgctggctgcaggactgccgctccaggcggccaagcggttccgtgatgtgctgggccatgagcagtatccggatcacatgagggagaacaaccaattacgtggctggtcttcagatgaaaatgaatgggatgaacagctgtatccagtgtggaggaggggagagggcagatggaaggactcctgggaaggaggccgtgtgcaggcagccctaaccagtgattcaccggccttggtgggttccaatatcaccttcgtagtgaacctggtgttccccagatgccagaaggaagatgccaacggcaatatcgtctatgagaggaactgcagaagtgatttggagctggcttctgacccgtatgtctacaactggaccacaggggcagacgatgaggactgggaagacagcaccagccaaggccagcacctcaggttccccgacgggaagcccttccctcgcccccacggacggaagaaatggaacttcgtctacgtcttccacacacttggtcagtattttcaaaagctgggtcggtgttcagcacgagtttctataaacacagtcaacttgacagttggccctcaggtcatggaagtgattgtctttcgaagacacggccgggcatacattcccatctccaaagtgaaagacgtgtatgtgataacagatcagatccctatattcgtgaccatgtaccagaagaatgaccggaactcgtctgatgaaaccttcctcagagacctccccattttcttcgatgtcctcattcacgatcccagtcatttcctcaactactctgccatttcctacaagtggaactttggggacaacactggcctgtttgtctccaacaatcacactttgaatcacacgtatgtgctcaatggaaccttcaactttaacctcaccgtgcaaactgcagtgccgggaccatgcccctcacccacaccttcgccttcttcttcgacttctccttcgcctgcatcttcgccttcacccacattatcaacacctagtccctctttaatgcctactggccacaaatccatggagctgagtgacatttccaatgaaaactgccgaataaacagatatggttacttcagagccaccatcacaattgtagatggaatcctagaagtcaacatcatccaggtagcagatgtcccaatccccacaccgcagcctgacaactcactgatggacttcattgtgacctgcaaaggggccactcccacggaagcctgtacgatcatctctgaccccacctgccagatcgcccagaacagggtgtgcagcccggtggctgtggatgagctgtgcctcctgtccgtgaggagagccttcaatgggtccggcacgtactgtgtgaatttcactctgggagacgatgcaagcctggccctcaccagcgccctgatctctatccctggcaaagacctaggctcccctctgagaacagtgaatggtgtcctgatctccattggctgcctggccatgtttgtcaccatggttaccatcttgctgtacaaaaaacacaagacgtacaagccaataggaaactgcaccaggaacgtggtcaagggcaaaggcctgagtgtttttctcagccatgcaaaagccccgttctcccgaggagaccgggagaaggatccactgctccaggacaagccatggatgctctaagtcttcactctcacttctgactgggaacccactcttctgtgcatgtatgtgagctgtgcagaagtacatgactggtagctgttgttttctacggattattgtaaaatgtatatcatggtttagggagcgtagttaattggcattttagtgaagggatgggaagatagtatttcttcacatctgtattgtggtttttatattgttaatagggtgggcacattgtgtctgaagggggagggggaggtcactgctacttaaggtcctaggttaactgggagaggatgccccaggctccttagatttctacacaagatgtgcctgaacccagctagtcctgacctaaaggccatgctttatcaactctatctcagctcattgaacatacctgagcacctgatggaattataatggaaccaagcttgttgtatggtgtgtgtgtgtatataagatactcattaaaaagatagtctattaaaaaaaaaaaaaaaaaaaaaaaaaaaaaaaaa

SEQ ID NO: 10 - protein, 572 aa, Rattus norvegicus
MESLCGVLVFLLLAAGLPLQAAKRFRDVLGHEQYPDHMRENNQLRGWSSDENEWDEQLYPVWRRGEGRWKDSWEGGRVQAALTSDSPALVGSNITFVVNLVFPRCQKEDANGNIVYERNCRSDLELASDPYVYNWTTGADDEDWEDSTSQGQHLRFPDGKPFPRPHGRKKWNFVYVFHTLGQYFQKLGRCSARVSINTVNLTVGPQVMEVIVFRRHGRAYIPISKVKDVYVITDQIPIFVTMYQKNDRNSSDETFLRDLPIFFDVLIHDPSHFLNYSAISYKWNFGDNTGLFVSNNHTLNHTYVLNGTFNFNLTVQTAVPGPCPSPTPSPSSSTSPSPASSPSPTLSTPSPSLMPTGHKSMELSDISNENCRINRYGYFRATITIVDGILEVNIIQVADVPIPTPQPDNSLMDFIVTCKGATPTEACTIISDPTCQIAQNRVCSPVAVDELCLLSVRRAFNGSGTYCVNFTLGDDASLALTSALISIPGKDLGSPLRTVNGVLISIGCLAMFVTMVTILLYKKHKTYKPIGNCTRNVVKGKGLSVFLSHAKAPFSRGDREKDPLLQDKPWML